(19) United States
(12) Patent Application Publication
(10) Pub. No.: US 2022/0384042 A1
(43) Pub. Date: Dec. 1, 2022

(54) Deep Learning System for Diagnosis of Chest Conditions from Chest Radiograph

(71) Applicant: Google LLC, Mountain View, CA (US)

(72) Inventors: Andrew Beckmann Sellergren, El Cerrito, CA (US); Shravya Ramash Shetty, San Francisco, CA (US); Siddhant Mittal, New York, NY (US); David Francis Steiner, Redwood City, CA (US); Anna Majkowska, San Francisco, CA (US); Gavin Elliott Duggan, Mountain View, CA (US)

(21) Appl. No.: 17/775,139

(22) PCT Filed: Oct. 13, 2020
(86) PCT No.: PCT/US2020/055365

§ 371 Date: May 6, 2022

Related U.S. Application Data
(60) Provisional application No. 62/931,974, filed on Nov. 7, 2019.

(51) Int. Cl. G16H 50/20; G16H 30/20

(52) U.S. Cl. CPC G16H 50/20; G16H 30/20

(57) ABSTRACT

The present disclosure provides systems and methods for training and/or employing machine-learned models (e.g., artificial neural networks) to diagnose chest conditions such as, as examples, pneumothorax, opacity, nodules or masses, and/or fractures based on chest radiographs. For example, one or more machine-learned models can receive and process a chest radiograph to generate an output. The output can indicate, for each of one or more chest conditions, whether the chest radiograph depicts the chest conditions (e.g., with some measure of confidence). The output of the machine-learned models can be provided to a medical professional and/or patient for use in providing treatment to the patient (e.g., to treat a detected condition).

### RELATED APPLICATIONS

This application claims priority to and the benefit of U.S. Provisional Patent Application No. 62/931,974, filed Nov. 7, 2019. U.S. Provisional Patent Application No. 62/931,974 is hereby incorporated herein by reference in its entirety.

### FIELD

The present disclosure relates generally to diagnostic technology. More particularly, the present disclosure relates to using deep learning models to diagnose chest conditions such as, for example, pneumothorax, opacity, nodules or masses, and/or fractures based on chest radiographs.

### BACKGROUND

Despite being one of the most common and well established imaging modalities, radiography is subject to significant inter-reader variability and suboptimal sensitivity for the detection of important clinical findings. Thus, even among a group of persons trained to interpret radiographs (e.g., radiologists), there may be significant disagreement among the correct interpretation, including instances where a majority of the group fails to detect a challenging but critical condition.

### SUMMARY

Aspects and advantages of embodiments of the present disclosure will be set forth in part in the following description, or can be learned from the description, or can be learned through practice of the embodiments.

One example aspect of the present disclosure is directed to a method for improved interpretation of chest radiographs via machine learning. The method includes obtaining, by one or more computing devices, data descriptive of one or more machine-learned models configured to receive and process a chest radiograph to generate an output that indicates whether the chest radiograph depicts one or more chest conditions. The method includes accessing, by the one or more computing devices, a training dataset comprising a plurality of training examples, wherein each of the plurality of training examples comprises an example chest radiograph and a label assigned to the example chest radiograph that indicates whether the example chest radiograph depicts the one or more chest conditions. For at least some of the plurality of training examples, the label assigned to the example chest radiograph comprises an adjudicated label generated based on a plurality of final evaluations respectively provided for the example chest radiograph by a plurality of human evaluators. Prior to providing the plurality of final evaluations, the human evaluators were provided, via one or more rounds of intermediate evaluation, with one or more respective intermediate evaluations provided by the other human evaluators. The method includes training, by the one or more computing devices, the one or more machine-learned models using the plurality of training examples included in the training dataset.

Another example aspect of the present disclosure is directed to a method for generating improved training data for machine-learned models configured to receive and process a chest radiograph to generate an output that indicates whether the chest radiograph depicts one or more chest conditions. The method is performed for one or more of a plurality of training examples that respectively comprise a plurality of example chest radiographs. The method includes providing the example chest radiograph to a plurality of human evaluators. The method includes receiving a plurality of intermediate evaluations for the example chest radiograph respectively from the plurality of human evaluators. The method includes, for each of one or more rounds of intermediate evaluation: providing the plurality of intermediate evaluations to each of the plurality of human evaluators; and receiving an indication for each of the plurality of human evaluators of whether such human evaluator maintains or changes their respective intermediate evaluation. The method includes, after the one or more rounds of intermediate evaluation, determining a plurality of final evaluations for the example chest radiograph respectively for the plurality of human evaluators. The method includes generating a label for the example chest radiograph based on the plurality of final evaluations. The method includes storing the label with the example chest radiograph in a training dataset.

Another example aspect of the present disclosure is directed to a method for performing inverse probability weighting when evaluating machine-learned model performance on chest radiographs. The method is performed for one or more of a plurality of reference examples included in a reference dataset. The method includes obtaining, by one or more computing devices, an output generated by one or more machine-learned models for a reference chest radiograph, wherein the output indicates whether the reference chest radiograph depicts one or more chest conditions. The method includes accessing, by the one or more computing devices, a label associated with the reference chest radiograph. The method includes evaluating, by the one or more computing devices, a weighted performance of the one or more machine-learned models for the reference chest radiograph based at least in part on a comparison of the output to the label, wherein the weighted performance is weighted using a weight value that is inversely proportional to an amount of enrichment associated with the reference example.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIGS. 1A-C depict example computing systems according to example embodiments of the present disclosure.

FIG. 2 depicts an example process for obtaining an adjudicated label according to example embodiments of the present disclosure.

FIG. 3 depicts a block diagram of an example technique to determine a weighted performance evaluation of a trained model according to example embodiments of the present disclosure.

FIG. 4 depicts a block diagram of an example technique to use a weighted loss function to train a model according to example embodiments of the present disclosure.

FIG. 5 depicts a block diagram of a multi-headed model configured to produce multiple radiological inferences according to example embodiments of the present disclosure.

FIG. 6 depicts a flow chart diagram of an example method to obtain and use an adjudicated label according to example embodiments of the present disclosure.

FIG. 7 depicts a flow chart diagram of an example method to determine a weighted performance evaluation of a model output according to example embodiments of the present disclosure.

### DETAILED DESCRIPTION

Generally, the present disclosure is directed to systems and methods for training and/or employing machine-learned models (e.g., artificial neural networks) to diagnose chest conditions such as, as examples, pneumothorax, opacity, nodules or masses, and/or fractures based on chest radiographs. For example, one or more machine-learned models can receive and process a chest radiograph to generate an output. The output can indicate, for each of one or more chest conditions, whether the chest radiograph depicts the chest conditions (e.g., with some measure of confidence). The output of the machine-learned models can be provided to a medical professional and/or patient for use in providing treatment to the patient (e.g., to treat a detected condition).

One aspect of the present disclosure is directed to training the machine-learned models described herein using a training dataset that includes adjudicated labels as the reference standard. Use of adjudicated training data can improve the accuracy of the resulting models, particularly in the case of challenging diagnoses which may be missed by a majority of evaluators.

More particularly, a critical aspect of developing clinically relevant diagnostic models involves training and evaluation of the models on reference data sets that have defined “ground truth” labels. However, inter-reader variability in establishing these reference standard image labels has a significant impact on performance and evaluation.

In particular, prior work in deep learning for radiographic image analysis has generally utilized a single reader or a majority-vote approach across multiple independent readers to provide reference standard labels. However, due to errors or inconsistencies in the resulting labels, such approaches may lead to overestimation of model performance. For example, challenging but critical findings may be under-recognized and thus mislabeled by a majority-vote approach if they are only (correctly) identified by a minority of the independent readers. This can result in the inability for a model to detect these findings (due to incorrect training labels), but also the inability to measure these errors (due to incorrect reference standard labels), resulting in a false sense of model accuracy.

To resolve these issues, the present disclosure provides an improved process for adjudication of ground truth reference labels by human evaluators. In particular, the present disclosure provides an adjudication process in which multiple (e.g., 3, 5, etc.) human evaluators (e.g., radiologists) are able to collaboratively assess a reference radiograph (e.g., a reference chest radiograph) to generate an adjudicated label for the reference radiograph. Specifically, the adjudication process can occur over one or more rounds of intermediate evaluation (e.g., 2 rounds, 3 rounds, 5 rounds, etc.). At each round of intermediate evaluation, each human evaluator can be given the opportunity to review the reference example and to provide a respective intermediate evaluation.

According to an aspect of the present disclosure, at each round of intermediate evaluation, each human evaluator can also be provided with the opportunity to review the intermediate evaluations provided by the other evaluators in the previous and/or current round(s). Each evaluator can decide whether to maintain or update their own respective intermediate evaluation based on the information about other, potentially differing viewpoints.

By providing the ability for the human evaluators to consider the other evaluators' evaluations, the human evaluators may be able to identify a condition that they previously failed to detect. Stated differently, certain conditions detectable via radiology may be significantly challenging, such that even a majority of reviewers fail to correctly diagnose the condition. However, in the proposed scheme in which collaborative discussion/consideration can occur over one or more rounds, the minority viewpoint may be considered before a final judgment. In cases where the minority viewpoint is in fact the correct diagnosis, the discussion may enable the minority members to persuade the majority members to change their diagnosis. For example, one astute expert evaluator may be able to convince the other evaluators that they originally failed to provide the correct diagnosis. In such fashion, the evaluations provided by the human evaluators can provide more accurate labels for highly challenging cases.

In some implementations, at each round of intermediate evaluation, the human evaluators can provide respective written commentary on their respective intermediate evaluations to the other human evaluators. For example, written notes can be sent from each evaluator to the group. This can allow a human evaluator to provide a written description of why they provided their respective evaluation and, potentially, why it is superior to contrary evaluations.

Similarly, in some implementations, at each round of intermediate evaluation, the human evaluators can provide respective visual markup on the example chest radiograph to the other human evaluators. For example, the visual markup can include coloration, annotation, and/or other forms of markup that the evaluator can use to make a visual case for the evaluation.

In some implementations, at each round of intermediate evaluation, some or all of the human evaluators can be anonymous to the other human evaluators. By keeping the identity of the evaluators anonymous, the other evaluators can be prevented from allowing political, social, or other implicit biases to affect how much deference is given to the other evaluators' evaluations. For example, if one of the human evaluators is a preeminent radiologist, keeping her identity secret will prevent the other evaluators from simply deferring to her judgment out of respect or other concerns.

In some implementations, the rounds of intermediate evaluation can be performed synchronously, such that the evaluators are able to simultaneously collaborate (e.g., via a chat interface, video conference, etc.). Alternatively or additionally, the rounds of intermediate evaluation can be performed asynchronously. An asynchronous process can enable evaluators to label images on a flexible schedule, avoiding the need to align multiple clinical schedules.

Following the one or more rounds of intermediate evaluation (e.g., as soon as a consensus is achieved or a maximum number of rounds is reached), each human evaluator can provide a final evaluation. For example, the final evaluation can simply be the last intermediate evaluation that was provided in the last round of intermediate evaluation. The final evaluations from the multiple human evaluators can be combined or aggregated to generate an adjudicated label for the reference radiograph. For example, a voting scheme can be applied to select as the adjudicated label the condition evaluation that was provided by a majority of evaluators.

The proposed adjudication process produces adjudicated labels (e.g., useful for training, testing, and/or validation) which exhibit improved accuracy, particularly in challenging but critical edge cases. By providing labels with improved accuracy, the resulting machine-learned models which learn from such labels can also exhibit improved accuracy. Furthermore, the performance of models tested on such labels can be accurately measured.

Another aspect of the present disclosure is directed to the evaluation of the machine-learned models described herein using a population-adjusted evaluation approach which accounts for enrichment of positive findings within the reference dataset (e.g., training dataset, testing dataset, validation dataset, etc.).

More particularly, dataset selection is an important element of machine learning approaches in radiology. Enrichment for positive findings is a strategy in creating datasets that can provide requisite examples for training and evaluation with efficient use of labelling resources. In particular, in dataset enrichment, training examples which have positive training labels (e.g., depict a condition to be detected) are over-represented within the reference dataset, thereby providing the model additional opportunities to learn or test upon the positive label, which might otherwise be very rare (e.g., if the condition to be detected occurs only rarely within the general population).

However, because the enriched dataset does not necessarily reflect real-world prevalence or case-mix diversity, such enrichment can also prevent meaningful clinical interpretation of diagnostic performance. Taken together, issues of enrichment and poor case-mix diversity can degrade the meaningfulness of commonly reported performance metrics for machine learning systems.

To combat this challenge, the present disclosure provides improved techniques for evaluation of machine-learned models which account for enrichment of the reference dataset(s). In particular, at each instance in which a model's performance is evaluated (e.g., during training, testing, or validation), a raw performance score for the model (e.g., the score that would normally be generated) can be modified with a weight value, where the weight value is inversely proportional to an amount of enrichment that was performed on the example against which the model's performance is being evaluated.

Briefly, based on various selection criteria, each reference example (e.g., training example or test example) can be assigned to an “enrichment group” to facilitate weighting. As one example, the groups can be defined by or coextensive with the labels assigned to the reference examples (e.g., all reference examples that have a label of ‘yes’ for the condition of ‘fracture’ can be assigned to one group). As another example, the groups can be based on a level of confidence associated with the respective labels (e.g., highly confident in a positive diagnosis vs. simply abnormal vs. highly confident in a negative diagnosis).

In some implementations, to calculate the weight for a particular reference example, a computing system can evaluate how often members of the group appear in the reference dataset (e.g., training dataset or test dataset) vs. how often members of the group appear in a parent dataset. For example, the parent dataset can include all known reference examples. For example, the parent dataset can exhibit a population-level distribution.

More particularly, in one example, the weight for each reference example can equal the number of examples included in the parent dataset included in the grouping associated with the reference example divided by the number of reference examples included in the reference dataset and included in the grouping associated with the reference example. To provide an example, if the parent dataset includes 20 examples that are included in a same selection group (e.g., have the same label) while the reference dataset includes only 10 examples, then the weight value for each of the 10 examples can equal 2. Thus, the weight is inversely proportional to the “amount of enrichment” with the lowest possible weight of 1 corresponding to the scenario when all possible images of a label-type are included in the enriched set. (e.g., these are the relatively rare image-types that are highly enriched in the reference set relative to the actual clinical case-mix and thus the low weight reflects that they are rare during adjustment).

The weighted performance evaluation described above can be applied during training and/or post-training evaluation (e.g., testing). For example, during training, the weight value can be applied as part of a loss function to control how much affect the loss function has on updating the model parameters. During testing, the weight value can be applied to performance measures such as accuracy measures to obtain a more accurate measurement of the model's true performance when applied to cases with a population-level distribution (e.g., as opposed to enriched distributions exhibited by specialized reference datasets).

As demonstrated in U.S. Provisional Patent Application No. 62/931,974, example models trained according to the techniques described herein achieved parity to chest x-ray interpretations of board-certified radiologists for the detection of pneumothorax, nodules/mass, opacity, and fracture on a diverse, multi-center chest x-ray data set. In particular, the example experimental data contained U.S. Provisional Patent Application No. 62/931,974 demonstrates differences in reference standard methodologies and resulting effects on performance evaluation, reinforcing the importance of rigorous and standardized methods to facilitate the development of artificial intelligence applications in radiology.

Although example aspects of the present disclosure focus on processes for generating adjudicated labels for radiographs (and chest radiographs in particular) the adjudication process can be performed to generate adjudicated labels for other forms of modalities of training examples. Furthermore, although example aspects of the present disclosure focus on determining weighted performance evaluation for radiological inferences, the weighted performance evaluation can be applied to measure the performance of other forms of inferences supplied by machine-learned models. As one example, while example aspects focus on chest radiographs and chest conditions, the techniques described herein are extensible to a radiograph of any portion of a human body (e.g., hand) and to any condition detectable from such radiograph (e.g., fracture). Likewise, the techniques described herein are extensible to other forms of medical imaging (e.g., CT scan) and to any conditions detectable from such forms of medical imaging (e.g., brain damage).

In some implementations, the data used by the models (e.g., for training and/or inference) can be de-identified data. For example, personally identifiable information, such as location, name, exact birth date, contact information, biometric information, facial photographs, etc. can be scrubbed from the records prior to being transmitted to and/or utilized by the models and/or a computing system including the models. For example, the data can be de-identified to protect identity of individuals and to conform to regulations regarding medical data, such as HIPAA, such that no personally identifiable information (e.g., protected health information) is present in the data used by the models and/or used to train the models.

Further to the descriptions above, a user may be provided with controls allowing the user to make an election as to both if and when systems, programs, or features described herein may enable collection of user information (e.g., observations, interventions, states, etc.). In addition, certain data may be treated in one or more ways before it is stored or used, so that personally identifiable information is removed. For example, a user's identity may be treated so that no personally identifiable information can be determined for the user. Thus, the user may have control over what information is collected about the user, how that information is used, and what information is provided to the user.

For instance, a patient may be provided with controls allowing the patient to consent to use of the patient's electronic medical record (EMR) data. As another example, the patient may be provided with controls allowing the patient to restrict some or all forms of EMR data from being collected or stored. As another example, the patient may be provided with controls allowing the patient to limit the use or continued use of the EMR data, such as by restricting the EMR data from being used as training data or for a prediction associated with a different patient. For instance, machine-learned models can be trained using only publicly available datasets of scrubbed and de-identified data (e.g., using no protected health information derived from patients).

With reference now to the Figures, example embodiments of the present disclosure will be discussed in further detail.

### Example Devices and Systems

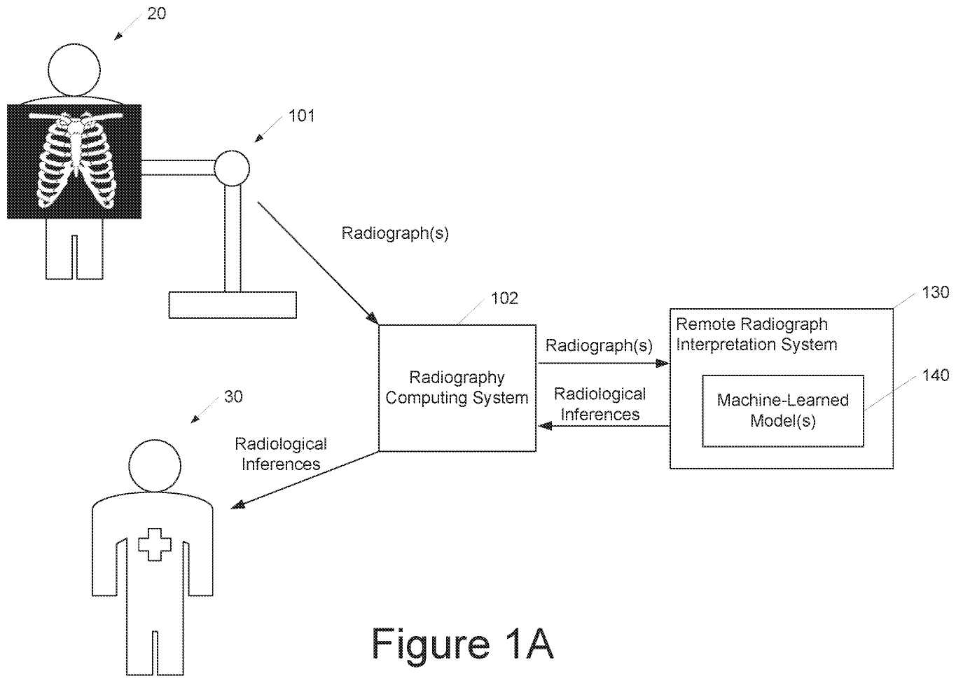

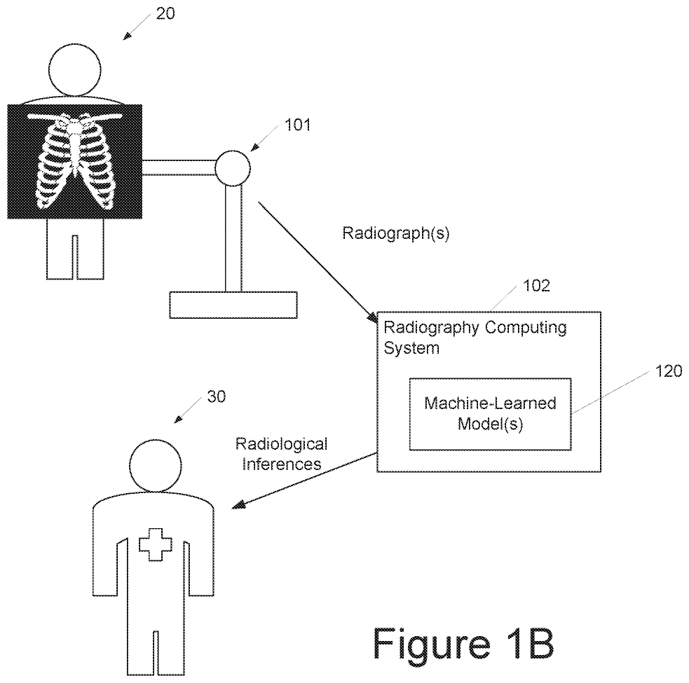

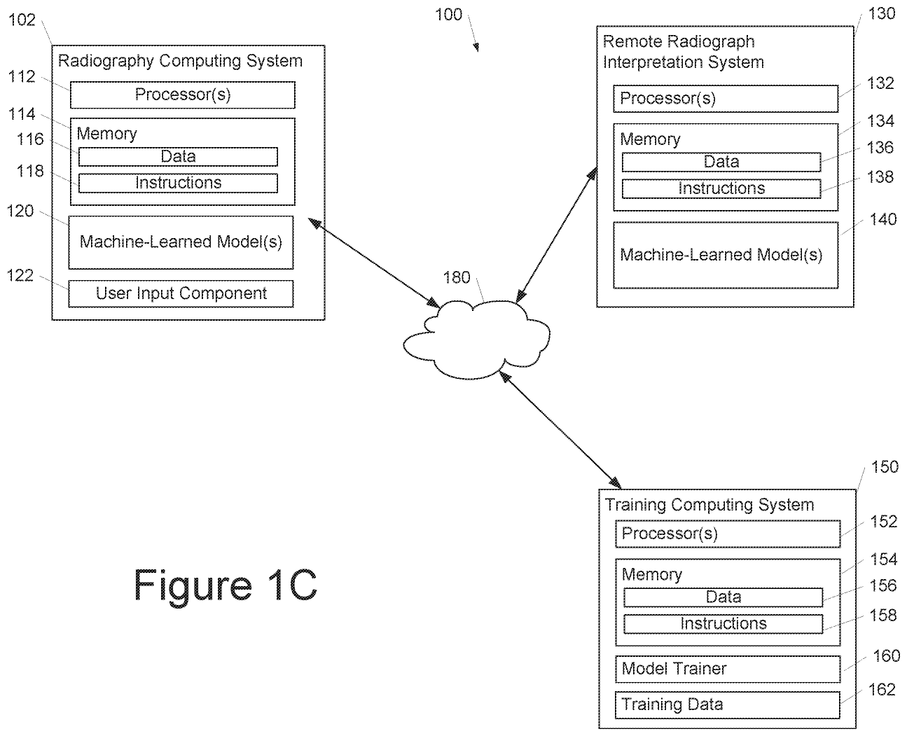

FIGS. 1A-C depict example computing systems according to example embodiments of the present disclosure. In particular, FIG. 1A shows an example system where one or more machine-learned models 140 are employed by a remote radiograph interpretation system 130 to generate radiological inferences from radiographs generated by an x-ray machine 101. FIG. 1B shows an alternative system where one or more machine-learned models 120 are employed by a radiography computing system 102 to generate the radiological inferences. FIG. 1C shows components of the systems/devices as connected to enable training of the machine-learned models 120 or 140.

More particularly, referring first to FIGS. 1A and 1B, an x-ray machine 101 can be operated to generate one or more radiographs that depict portions of a patient 20. The radiographs can be initially collected or provided to a radiography computing system 102. For example, the radiography computing system 102 can be a computing system that is on-site with the x-ray machine 101. For example, the radiography computing system 102 can be a portion of the x-ray machine 101 (e.g., the radiography computing system 102 can control the x-ray machine 101 and receive and store the x-ray data (e.g., radiographs) from the x-ray machine 101 upon capture). Alternatively or additionally, the radiography computing system 102 can be a separate system that is on-site at a medical care facility along with the x-ray machine 101. For example, the radiography computing system 102 can be a medical provider's computing system such as a computing system operated for a hospital, physician's office, and/or the like (e.g., which stores various types of patient files or data).

In FIG. 1A, the radiographs are transmitted from the radiography computing system 102 to a remote radiograph interpretation system 130. For example, the remote radiograph interpretation system 130 can be a cloud service (e.g., accessible via API(s)) to which the radiography computing system 102 can make calls to receive radiological inferences. Specifically, the remote radiograph interpretation system 130 can store and use one or more machine-learned models 140 to generate one or more radiological inferences based on the radiograph(s). For example, each radiological inference can indicate the presence or absence (e.g., with some measure of confidence) of whether a given radiograph depicts a given condition. The remote radiograph interpretation system 130 can transmit the radiological inference(s) to the radiography computing system 102 and the radiography computing system 102 can provide (e.g., display) the radiological inferences to a care provider 30 (e.g., physician or other medical professional). The care provider 30 can use the radiological inferences (e.g., in addition to their own judgment) to determine a diagnosis and/or treatment plan for the patient 20. In some implementations, the radiological inferences can specifically include a suggested treatment for the inferred conditions.

FIG. 1B is highly similar to FIG. 1A, except that the radiographs are analyzed locally at the radiography computing system 102 using one or more machine-learned models 120 which are locally stored at the radiography computing system 102.

Referring now to FIG. 1C, a system 100 for enabling the machine-learned models 120 and 140 includes the radiography computing system 102, the remote radiograph interpretation system 130, and a training computing system 150 that are communicatively coupled over a network 180.

The radiography computing system 102 can include any type of computing device, such as, for example, a personal computing device (e.g., laptop or desktop), a mobile computing device (e.g., smartphone or tablet), an embedded computing device, one or more servers, a device contained within an x-ray machine, or any other type of computing device.

The radiography computing system 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the radiography computing system 102 to perform operations.

In some implementations, the radiography computing system 102 can store or include one or more machine-learned models 120. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks, or other forms of neural networks.

In some implementations, the one or more machine-learned models 120 can be received from the remote radiograph interpretation system 130 over network 180, stored in the radiography computing system memory 114, and then used or otherwise implemented by the one or more processors 112. In some implementations, the radiography computing system 102 can implement multiple parallel instances of a single machine-learned model 120 (e.g., to perform parallel radiological inference across multiple instances of radiographs).

Additionally or alternatively, one or more machine-learned models 140 can be included in or otherwise stored and implemented by the remote radiograph interpretation system 130 that communicates with the radiography computing system 102 according to a client-server relationship. For example, the machine-learned models 140 can be implemented by the remote radiograph interpretation system 140 as a portion of a web service (e.g., a radiology service). Thus, one or more models 120 can be stored and implemented at the radiography computing system 102 and/or one or more models 140 can be stored and implemented at the remote radiograph interpretation system 130.

The radiography computing system 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The remote radiograph interpretation system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the remote radiograph interpretation system 130 to perform operations.

In some implementations, the remote radiograph interpretation system 130 includes or is otherwise implemented by one or more server computing devices. In instances in which the remote radiograph interpretation system 130 includes plural server computing devices, such server computing devices can operate according to sequential computing architectures, parallel computing architectures, or some combination thereof.

As described above, the remote radiograph interpretation system 130 can store or otherwise include one or more machine-learned models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks.

The radiography computing system 102 and/or the remote radiograph interpretation system 130 can train the models 120 and/or 140 via interaction with the training computing system 150 that is communicatively coupled over the network 180. The training computing system 150 can be separate from the remote radiograph interpretation system 130 or can be a portion of the remote radiograph interpretation system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the radiography computing system 102 and/or the remote radiograph interpretation system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the machine-learned models 120 and/or 140 based on a set of training data 162. The training data 162 can include, for example, example training or reference radiographs that have been labeled with one or more adjudicated labels. For example, the example radiographs can be chest radiographs. The adjudicated labels for each radiograph can indicate (e.g., with some confidence, which may be binary or continuous) whether the radiograph depicts one or more conditions.

As more specific examples, example training datasets can be as follows: a first Dataset 1 (DS1) can include 759,611 de-identified frontal chest radiographs (digital and scanned) with reports from 538,390 patients. This dataset consists of all consecutive inpatient and outpatient images in DICOM format obtained from 5 regional centers of Apollo Hospitals group in 5 cities in India (Bangalore, Bhubaneswar, Chennai, Hyderabad, New Delhi) between November 2010 and January 2018. A second dataset can be the publicly available dataset from the National Institutes of Health (ChestX-ray14)(18,21) consisting of 112,120 frontal chest radiograph images from 30,805 patients (Table 1). Because DS1 includes all chest x-rays from multiple different hospitals, the abnormalities in this data set reflect the natural population prevalence of different abnormalities in these populations. In contrast, ChestX-ray14 is enriched for various thoracic abnormalities relative to the general population.

One example process for preparing the training dataset is as follows: For DS1, patients can be randomly assigned to training, tuning/validation, or testing sets. For ChestX-ray14, the original test set of 25,596 images from 2,797 patients can be preserved. The remaining 86,524 images from 28,008 patients can be randomly split into training (80%) and tuning/validation sets (20%). For both datasets, images from the same patient can remain in the same split to avoid training and testing on the same patients.

As further examples, to provide a sufficient number of diverse and high quality labeled images with positive findings, approximately 2,000 images from both DS1 and ChestX-ray14 can be selected. Because ChestXray14 is already enriched for positive findings, images can be selected at random from the available images. For DS1, images can be selected based on radiology reports to enrich for positive findings while maintaining case-mix diversity and also allowing population-adjustment at analysis by inverse probability weighting. Though the radiology reports can be used to facilitate case enrichment, the reference standard labels for each image can be provided via adjudicated radiologist image review.

In some example implementations, training examples can be labeled via two approaches, expert image annotation and natural language processing (NLP). For example, to label training images (e.g., DS1 images), an NLP model can be used to predict image labels from original radiology reports using approximately 35,000 reports. Briefly, a one-dimensional deep convolutional neural network can be trained, and performance can be evaluated against human labeled reports. The train, validation, and test sets for NLP model development can be subsets of the corresponding data splits used for image modeling.

In some implementations, if the user has provided consent, the training examples can be provided by the radiography computing system 102. Thus, in such implementations, the model 120 provided to the radiography computing system 102 can be trained by the training computing system 150 on user-specific data received from the radiography computing system 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 1C illustrates one example computing system that can be used to implement the present disclosure. Other computing systems can be used as well. For example, in some implementations, the radiography computing system 102 can include the model trainer 160 and the training dataset 162. In such implementations, the models 120 can be both trained and used locally at the radiography computing system 102. In some of such implementations, the radiography computing system 102 can implement the model trainer 160 to personalize the models 120 based on user-specific data.

### Example Label Adjudication Process

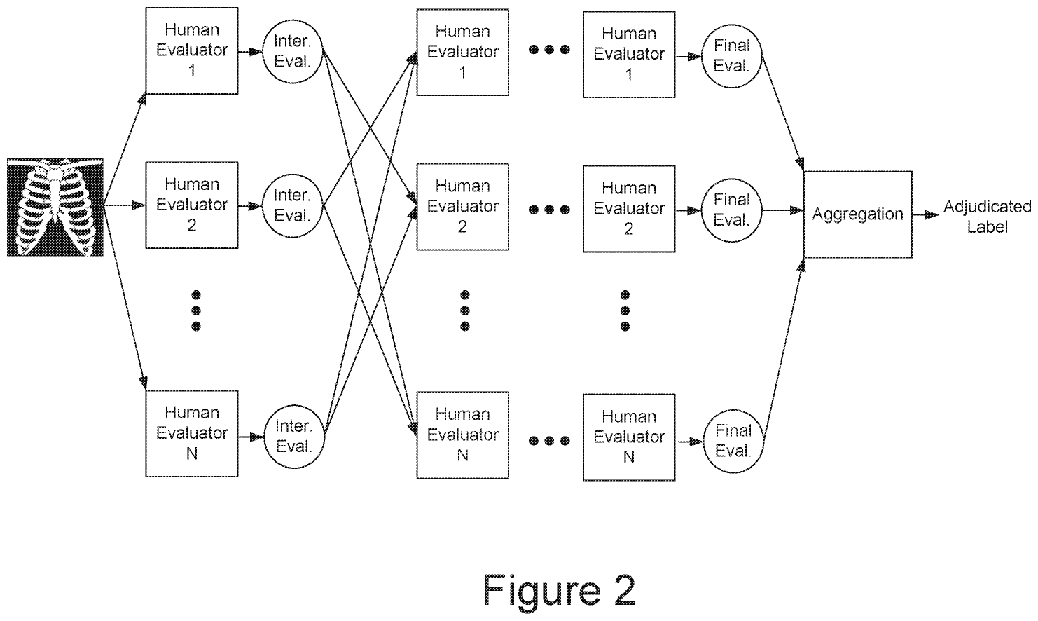

FIG. 2 depicts an example process for obtaining an adjudicated label according to example embodiments of the present disclosure. In particular, in the illustrated adjudication process, multiple (e.g., 1-N) human evaluators (e.g., radiologists) are able to collaboratively assess a reference radiograph (e.g., a reference chest radiograph) to generate an adjudicated label for the reference radiograph. Specifically, the adjudication process can occur over one or more rounds of intermediate evaluation (e.g., 2 rounds, 3 rounds, 5 rounds, etc.). At each round of intermediate evaluation, each human evaluator can be given the opportunity to review the reference example and to provide a respective intermediate evaluation.

According to an aspect of the present, at each round of intermediate evaluation, each human evaluator can also be provided with the opportunity to review the intermediate evaluations provided by the other evaluators in the previous and/or current round(s). Each evaluator can decide whether to maintain or update their own respective intermediate evaluation based on the information about other, potentially differing viewpoints.

By providing the ability for the human evaluators to consider the other evaluators' evaluations, the human evaluators may be able to identify a condition that they previously failed to detect. Stated differently, certain conditions detectable via radiology may be significantly challenging, such that even a majority of reviewers fail to correctly diagnose the condition. However, in the proposed scheme in which collaborative discussion/consideration can occur over one or more rounds, the minority viewpoint may be considered before a final judgment. In cases where the minority viewpoint is in fact the correct diagnosis, the discussion may enable the minority members to persuade the majority members to change their diagnosis. For example, one astute expert evaluator may be able to convince the other evaluators that they originally failed to provide the correct diagnosis. In such fashion, the evaluations provided by the human evaluators can provide more accurate labels for highly challenging cases.

In some implementations, at each round of intermediate evaluation, the human evaluators can provide respective written commentary on their respective intermediate evaluations to the other human evaluators. For example, written notes can be sent from each evaluator to the group. This can allow a human evaluator to provide a written description of why they provided their respective evaluation and, potentially, why it is superior to contrary evaluations.

Similarly, in some implementations, at each round of intermediate evaluation, the human evaluators can provide respective visual markup on the example chest radiograph to the other human evaluators. For example, the visual markup can include coloration, annotation, and/or other forms of markup that the evaluator can use to make a visual case for the evaluation.

In some implementations, at each round of intermediate evaluation, some or all of the human evaluators can be anonymous to the other human evaluators. By keeping the identity of the evaluators anonymous, the other evaluators can be prevented from allowing political, social, or other implicit biases to affect how much deference is given to the other evaluators' evaluations. For example, if one of the human evaluators is a preeminent radiologist, keeping her identity secret will prevent the other evaluators from simply deferring to her judgment out of respect or other concerns.

In some implementations, the rounds of intermediate evaluation can be performed synchronously, such that the evaluators are able to simultaneously collaborate (e.g., via a chat interface, video conference, etc.). Alternatively or additionally, the rounds of intermediate evaluation can be performed asynchronously. An asynchronous process can enable evaluators to label images on a flexible schedule, avoiding the need to align multiple clinical schedules.

Following the one or more rounds of intermediate evaluation (e.g., as soon as a consensus is achieved or a maximum number of rounds is reached), each human evaluator can provide a final evaluation. For example, the final evaluation can simply be the last intermediate evaluation that was provided in the last round of intermediate evaluation that occurred before a finality condition is triggered. The final evaluations from the multiple human evaluators can be combined or aggregated to generate an adjudicated label for the reference radiograph. For example, a voting scheme can be applied to select as the adjudicated label the condition evaluation that was provided by a majority of evaluators.

The proposed adjudication process produces adjudicated labels (e.g., useful for training, testing, and/or validation) which exhibit improved accuracy, particularly in challenging but critical edge cases. By providing labels with improved accuracy, the resulting machine-learned models which learn from such labels can also exhibit improved accuracy. Furthermore, the performance of models tested on such labels can be accurately measured.

A more detailed description of one example implementation of this process is as follows: an example adjudication process can seek to identify four chest radiographic findings: pneumothorax, opacity, nodule/mass (as a specific subtype of opacity) and fracture. Clinical definitions for these categories can be based on the Fleischner Society Glossary of Terms for Thoracic Imaging, except for osseous fracture which can be defined as visible rib, clavicle, humeral, or vertebral body fractures. For example, a nodule can be defined as less than 3 cm and mass as 3 cm or greater. The presence or absence of each of these findings can be labeled at the image level. Chest tube and fracture acuity labels can also be collected.

In some examples, reference standard labels for the final validation and test set images can be assigned via adjudicated review by three radiologists. For each image in the test set, three readers can be assigned from a cohort of 11 board-certified radiologists (experience range 3-21 years in general radiology with no thoracic experts, and including A.D.). The three readers for each image of the validation set can be selected from a cohort of 13 individuals, consisting of both board-certified radiologists (no thoracic experts) and residents.

Briefly, images can be independently evaluated by 3 readers allowing disagreements to be resolved via up to 5 rounds of asynchronous, anonymous discussion by the same readers, but not enforcing consensus. In cases where consensus was not reached, the majority vote can optionally be used. All readers can have access to the patient age and image view (PA vs AP), but no additional clinical or patient data. Nodule/mass and pneumothorax can be adjudicated as present, absent, or “hedge” (i.e., uncertain if present or absent) and opacity and fracture as present or absent. For evaluation, hedge can be considered to be positive with the rationale that a clinical hedge would prompt additional read, action, and/or clinical follow up.

### Example Performance Evaluation

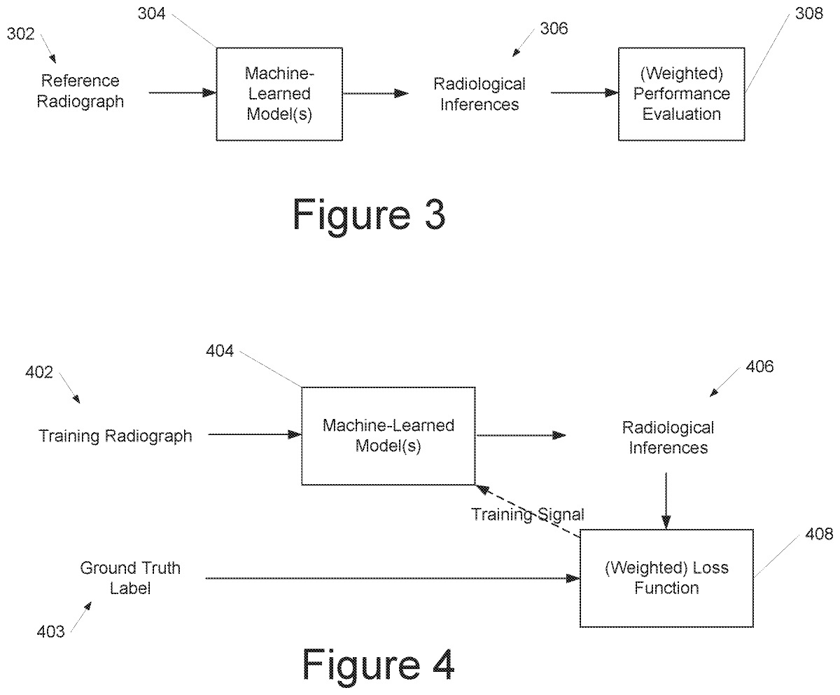

FIG. 3 depicts a block diagram of an example technique to determine a weighted performance evaluation of a trained model (e.g., during validation or testing) according to example embodiments of the present disclosure. In particular, as illustrated in FIG. 3, a machine-learned model 304 can receive and process a reference radiograph to generate one or more radiological inferences 306. A performance evaluation (e.g., a weighted performance evaluation) 308 can be performed on the one or more radiological inferences.

Specifically, when the performance of the model 304 is evaluated at 308, a raw performance score for the model 304 (e.g., an accuracy score or the like that would normally be generated using conventional evaluation techniques) can be modified with a weight value, where the weight value is inversely proportional to an amount of enrichment that was performed on the reference radiograph 302 against which the model's performance is being evaluated.

Briefly, based on various selection criteria, each reference example in a reference dataset (e.g., the reference radiograph 302) can be assigned to an “enrichment group” to facilitate weighting. As one example, the groups can be defined by or coextensive with the labels assigned to the reference examples (e.g., all reference examples that have a label of ‘yes’ for the condition of ‘fracture’ can be assigned to one group). As another example, the groups can be based on a level of confidence associated with the respective labels (e.g., highly confident in a positive diagnosis vs. simply abnormal vs. highly confident in a negative diagnosis).

In some implementations, to calculate the weight for the particular reference example 302, a computing system can evaluate how often members of the group appear in the reference dataset (e.g., training dataset or test dataset) vs. how often members of the group appear in a parent dataset. For example, the parent dataset can include all known reference examples. For example, the parent dataset can exhibit a population-level distribution that matches distributions of conditions in the population at large.

More particularly, in one example, the weight for the reference radiograph 302 can equal the number of examples included in the parent dataset included in the grouping associated with the reference radiograph 302 divided by the number of reference examples included in the reference dataset and included in the grouping associated with the reference radiograph 302. To provide an example, if the parent dataset includes 20 examples that are included in a same selection group (e.g., have the same label) while the reference dataset includes only 10 examples, then the weight value for each of the 10 examples can equal 2. Thus, the weight is inversely proportional to the “amount of enrichment” with the lowest possible weight of 1 corresponding to the scenario when all possible images of a label-type are included in the enriched set. (e.g., these are the relatively rare image-types that are highly enriched in the reference set relative to the actual clinical case-mix and thus the low weight reflects that they are rare during adjustment).

As one example, model performance can be evaluated by calculating the area under the receiver operating curve (AUC-ROC) using the per-image model prediction as the decision variable. Model performance can be compared to radiologist performance on the test sets at two operating points: the average radiologist sensitivity and the average radiologist specificity.

FIG. 4 depicts a block diagram of an example technique to use a weighted loss function to train a model according to example embodiments of the present disclosure. As illustrated in FIG. 4, a machine-learned model 404 can receive and process a training radiograph 402 to generate one or more radiological inferences 406. A loss function (e.g., a weighted loss function) 308 can compare (e.g., determine a difference between) the radiological inference(s) 406 and one or more ground truth labels 403 (e.g., adjudicated labels) to generate a loss value (e.g., a weighted loss value). Specifically, the weighted loss value for the training radiograph 402 can be weighted using a weight that is inversely proportional to an amount of enrichment associated with the training radiograph 402, as described herein. The loss value can be used as a training signal to train the machine-learned model(s) 404. For example, the weighted loss function 408 can be backpropagated through the model(s) 404 according to a gradient descent technique.

A more detailed description of an example process for evaluating model performance is as follows: Confidence intervals (CIs) for model and radiologist performance can be calculated using the nonparametric bootstrap method with 1,000-fold resampling at the image level. Model performance can be compared against radiologists using the Obuchowski-Rockette-Hillis procedure. Originally for comparing imaging modalities, this analysis has been adapted to comparison of radiologist performance to that of a standalone algorithm. For this analysis, the model can be thresholded using the operating point corresponding to the average radiologist sensitivity (when comparing specificity) and average radiologist specificity (when comparing sensitivity) and binarized agreement (i.e. correct vs. incorrect) was used for both model and radiologist. Noninferiority can be assessed by incorporating the margin parameter (5%) into the numerator of the test statistic. Briefly, a small p-value indicates that the null hypothesis (radiologists perform better than the model by 5% or more) is rejected. The jackknife method can be used to estimate the covariance terms for the test.

### Example Model Architectures

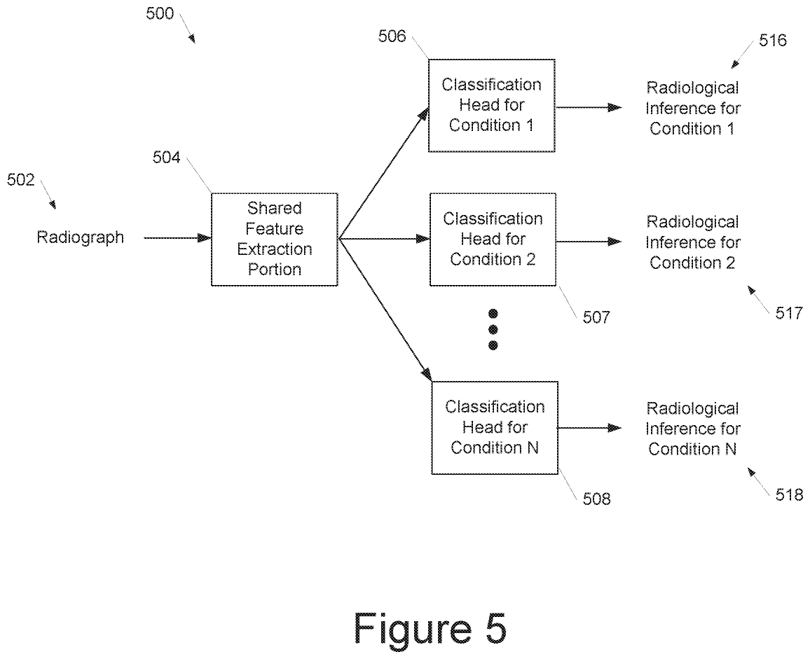

FIG. 5 depicts one example machine-learned model architecture that can be used. The architecture shown in FIG. 5 is just one example, other architectures can be used in addition or alternatively to the illustrated architecture.

As shown in FIG. 5, an example machine-learned model 500 can include a shared feature extraction portion 504 and a plurality of classification heads (e.g., heads 506, 507, 508) that provide respective radiological inferences (e.g., inferences 516, 517, 518) for different conditions. More particularly, the shared feature extraction portion 504 can receive a radiograph 502 as an input and can process the radiograph 502 to generate an intermediate representation, which can also be referred to as an embedding. The intermediate representation can, for example, be a continuous valued vector in a low- or high-dimensional latent space.

The shared feature extraction portion 504 can provide the intermediate representation to each classification head 506, 507, 508. Each classification head 506, 507, 508 can produce a respective radiological inference 516, 517, 518 based on the intermediate representation. Each respective inference 516, 517, 518 can be a binary inference (e.g., classification) or can be a continuous valued inference (e.g., in range [0,1]). A threshold can be applied to a continuous valued inference to obtain a binary inference, if desired. In some implementations, one or more of the outputs 516, 517, 518 can also indicate one or more saliency regions that were important for the corresponding prediction.

A more detailed description of example model architectures is as follows: Two separate deep learning models can be trained to distinguish the presence or absence of fracture and nodule/mass, respectively. A single deep learning model with two outputs can be trained to identify both pneumothorax and opacity. The models can be convolutional neural networks trained with the combined set of training images from both DS1 and ChestX-ray14 training sets. An Xception network can be used as the convolutional neural network architecture. The network can be pre-trained on 300 million natural images. For compatibility with the pretrained Xception architecture, the single channel grayscale image can be tiled to 3 channels (originally intended for RGB). The models can be trained with cross-entropy loss and Adam optimizer. The training can use an initial learning rate of 0.00143 with an exponential decaying rate of 0.865 and momentum with decaying rate of 0.822. using a decaying learning rate and momentum with a batch size of 16.

The models for ensembling can be selected based on the area under the precision-recall curve (AUC-PR) on the validation set. The final models can be an ensemble of multiple models trained on the same dataset and the final model predictions can be calculated as an average of the predictions of the ensemble.

Thus, as an example, for each condition, a multi-head model can be trained to optimize for a set of binary classification tasks that were empirically shown to improve performance for the condition of interest. Each training configuration can be run three times with identical parameters for model ensembling.

During training, the models can be saved as checkpoints periodically. Performance can be monitored on the validation sets of DS1 and ChestX-ray14, and the checkpoints with the highest AUC-PR on the condition of interest can be ensembled as the final model.

As one example, for pneumothorax, the model can be trained to predict presence of pneumothorax, airspace opacity, chest tube, and pneumothorax in the absence of a chest tube. One example final model is an ensemble based on the following checkpoints across the three training replicas: highest AUCPR on the pneumothorax task for DS1, highest AUC-PR on the pneumothorax task for ChestXray14, highest AUC-PR on the pneumothorax in the absence of chest tube task for DS1, and highest AUC-PR on the pneumothorax in the absence of chest tube task for ChestX-ray14. This results in 12 checkpoints.

As another example, for opacity, the same model training configuration can be used as for pneumothorax, but the airspace opacity output can be taken instead of the pneumothorax output. One example final model is an ensemble based on the following checkpoints across the three training replicas that had highest AUC-PR on the airspace opacity task for DS1 and highest AUC-PR on the airspace opacity task for ChestX-ray14. This results in 6 checkpoints.

As another example, for nodule/mass, the model can be trained to predict presence of nodule/mass, airspace opacity, nodule, and mass, and classify the count type of nodule (single, multiple, or diffuse). One example final model can be an ensemble based on checkpoints that had highest AUC-PR on the nodule/mass task for DS1 and highest AUC-PR on the nodule/mass task for ChestX-ray14, for each of the three training replicas, resulting in 6 checkpoints.

As another example, for fracture, the model can be trained to predict the presence of fracture and presence in each of the locations in left/right clavicle, left/right ribs, left/right shoulder, and spine. This allows predictions of multiple fractures across the various locations. One example final model is an ensemble based on checkpoints that had highest AUC-PR on the fracture task for DS1 and highest AUCPR on the fracture task for ChestX-ray14, for each of the three training replicas, resulting in 6 checkpoints.

### Example Methods

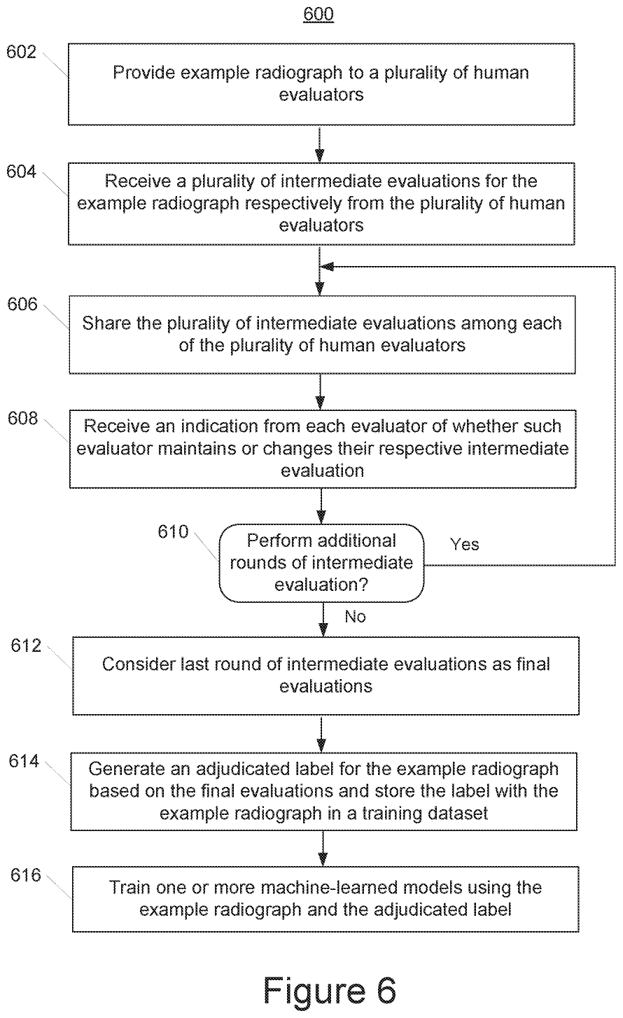

FIG. 6 depicts a flow chart diagram of an example method 600 to obtain and use an adjudicated label according to example embodiments of the present disclosure.

At 602, a computing system can provide an example radiograph to a plurality of human evaluators.

At 604, the computing system can receive a plurality of intermediate evaluations for the example radiograph respectively from the plurality of human evaluators.

At 606, the computing system can share the plurality of intermediate evaluations among each of the plurality of human evaluators.

At 608, the computing system can receive an indication from each evaluator of whether such evaluator maintains or changes their respective intermediate evaluation.

At 610, the computing system can determine whether additional rounds of intermediate evaluation should be performed. For example, a round counter can be compared to a maximum number of rounds (e.g., once 5 rounds are performed, the intermediate rounds end). As another example, the computing system can determine whether a consensus has been reached and, if so, the intermediate rounds can end.

If it is determined at 610 that an additional round of intermediate evaluation should be performed, then method 600 returns to 606 and again shares the current evaluations with all evaluators.

However, if it is determined at 610 that an additional round of intermediate evaluation should not be performed, then method 600 can proceed to 612. At 612, the computing system considers the last round of intermediate evaluations as final evaluations for the evaluators.

At 614, the computing system can generate an adjudicated label for the example radio graph based on the final evaluations and can store the label with the example radiograph in a training dataset.

At 616, the computing system can train one or more machine-learned models using the example radiograph and the adjudicated label.

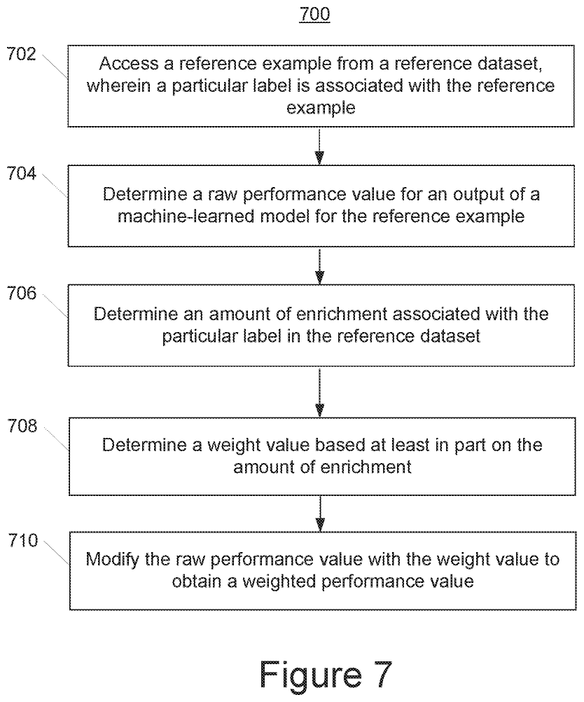

FIG. 7 depicts a flow chart diagram of an example method 700 to determine a weighted performance evaluation of a model output according to example embodiments of the present disclosure.

At 702, a computing system can access a reference example from a reference dataset. A particular label can be associated with the reference example.

At 704, the computing system can determine a raw performance value for an output produced by a machine-learned model based on the reference example.

At 706, the computing system can determine an amount of enrichment associated with the particular label in the reference dataset.

At 708, the computing system can determine a weight value based at least in part on the amount of enrichment.

At 710, the computing system can modify the raw performance value (e.g., through multiplication) with the weight value to obtain a weighted performance value.

### ADDITIONAL DISCLOSURE

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

In particular, although FIGS. 6 and 7 respectively depict steps performed in a particular order for purposes of illustration and discussion, the methods of the present disclosure are not limited to the particularly illustrated order or arrangement. The various steps of the methods 600 and 700 can be omitted, rearranged, combined, and/or adapted in various ways without deviating from the scope of the present disclosure.

Another example aspect of the present disclosure is directed to systems and methods that leverage machine-learned models for distinguishing normal versus abnormal chest radiographs. These systems, methods, and models have been demonstrated to generalize to unseen diseases. These systems, methods, and models can be used, in one example, as a triage tool.

More particularly, certain algorithms have been shown to detect specific findings, such as pneumonia, pleural effusion, and fracture, with comparable or higher performance than radiologists. However, by virtue of being developed to detect specific findings, these algorithms are unlikely to properly report other abnormalities that they were not trained to detect.

In view of the above, one example aspect of the present disclosure provides a machine learning system (e.g., a deep learning system) that classifies chest radiographs (CXRs) as normal or abnormal. In particular, in scenarios with a high reviewing burden for radiologists, these proposed systems and methods can be used to identify cases that are likely to contain findings and group them together for prioritized review, reducing the turnaround time for abnormal cases. Normal cases can also be quickly characterized by the AI algorithm, empowering healthcare professionals to quickly exclude certain differential diagnoses and allowing the diagnostic workup to proceed in other directions without delay.

These proposed systems can be used as a frontline point-of-care tool for non-radiologists. One advantage these proposed systems have over existing solutions is generalizability. By evaluating example implementations of the proposed system on six international datasets including two diseases it was not specifically trained to detect (e.g., tuberculosis and coronavirus disease 2019), it has been empirically shown that the proposed models perform well on a wider range of abnormalities than existing solutions.

1. A method for improved interpretation of chest radiographs via machine learning, the method comprising:
obtaining, by one or more computing devices, data descriptive of one or more machine-learned models configured to receive and process a chest radiograph to generate an output that indicates whether the chest radiograph depicts one or more chest conditions;
accessing, by the one or more computing devices, a training dataset comprising a plurality of training examples, wherein each of the plurality of training examples comprises an example chest radiograph and a label assigned to the example chest radiograph that indicates whether the example chest radiograph depicts the one or more chest conditions,
wherein, for at least some of the plurality of training examples, the label assigned to the example chest radiograph comprises an adjudicated label generated based on a plurality of final evaluations respectively provided for the example chest radiograph by a plurality of human evaluators, and
wherein, prior to providing the plurality of final evaluations, the human evaluators were provided, via one or more rounds of intermediate evaluation, with one or more respective intermediate evaluations provided by the other human evaluators; and
training, by the one or more computing devices, the one or more machine-learned models using the plurality of training examples included in the training dataset.

2. The method of claim 1, wherein, for at least one of the one or more rounds of intermediate evaluation, the plurality of human evaluators were provided respective written commentary on their respective intermediate evaluations to the other human evaluators.

3. The method of claim 1, wherein, for at least one of the one or more rounds of intermediate evaluation, the plurality of human evaluators were provided respective visual markup on the example chest radiograph to the other human evaluators.

4. The method of claim 1, wherein, for at least one of the one or more rounds of intermediate evaluation, each of the plurality of human evaluators was anonymous to the other human evaluators.

5. The method of claim 1, wherein each adjudicated label comprises a consensus or majority finding from the respective plurality of final evaluations respectively provided by the plurality of human evaluators.

6. The method of claim 1, further comprising, after training the one or more machine-learned models:
obtaining, by the one or more computing devices, a clinical chest radiograph associated with a patient; and
generating, by the one or more computing devices and using the one or more machine-learned models, a clinical diagnosis for the patient based on the clinical chest radiograph.

7. The method of claim 6, further comprising treating the patient based at least in part on the clinical diagnosis.

8. The method of claim 1, wherein at least some of the example chest radiographs included in the training dataset comprise frontal chest radiographs.

9. The method of claim 1, wherein the one or more chest conditions comprise one or more of: pneumothorax, opacity, nodule, and fracture.

10. The method of claim 1, wherein:
the label for each training example indicates the presence or absence of a plurality of chest conditions; and
the one or more machine-learned models comprise at least one multi-headed model that has a plurality of binary classification heads respectively for the plurality of chest conditions.

11. A method for generating improved training data for machine-learned models configured to receive and process a chest radiograph to generate an output that indicates whether the chest radiograph depicts one or more chest conditions, the method comprising:
for one or more of a plurality of training examples that respectively comprise a plurality of example chest radiographs:
providing the example chest radiograph to a plurality of human evaluators;
receiving a plurality of intermediate evaluations for the example chest radiograph respectively from the plurality of human evaluators;
for each of one or more rounds of intermediate evaluation:
providing the plurality of intermediate evaluations to each of the plurality of human evaluators; and
receiving an indication for each of the plurality of human evaluators of whether such human evaluator maintains or changes their respective intermediate evaluation;
after the one or more rounds of intermediate evaluation, determining a plurality of final evaluations for the example chest radiograph respectively for the plurality of human evaluators;
generating a label for the example chest radiograph based on the plurality of final evaluations; and
storing the label with the example chest radiograph in a training dataset.

12. The method of claim 11, wherein, for at least one of the one or more rounds of intermediate evaluation, providing the plurality of intermediate evaluations to each of the plurality of human evaluators comprises providing respective written commentary received from the plurality of human evaluators to the other human evaluators.

13. The method of claim 11, wherein, for at least one of the one or more rounds of intermediate evaluation, providing the plurality of intermediate evaluations to each of the plurality of human evaluators comprises providing respective visual markup on the chest radiograph received from the plurality of human evaluators to the other human evaluators.

14. The method of claim 11, wherein, for at least one of the one or more rounds of intermediate evaluation, each of the plurality of human evaluators is anonymous to the other human evaluators.

15. (canceled)

16. A method for performing inverse probability weighting when evaluating machine-learned model performance on chest radiographs, the method comprising:
for one or more of a plurality of reference examples included in a reference dataset:
obtaining, by one or more computing devices, an output generated by one or more machine-learned models for a reference chest radiograph, wherein the output indicates whether the reference chest radiograph depicts one or more chest conditions;
accessing, by the one or more computing devices, a label associated with the reference chest radiograph; and
evaluating, by the one or more computing devices, a weighted performance of the one or more machine-learned models for the reference chest radiograph based at least in part on a comparison of the output to the label, wherein the weighted performance is weighted using a weight value that is inversely proportional to an amount of enrichment associated with the reference example.

17. The method of claim 16, wherein:
the reference dataset comprises a subset of a parent dataset; and
the weight value for each reference example equals a number of examples included in the parent dataset included in a grouping associated with the reference example divided by a number of reference examples included in the reference dataset and included in the grouping associated with the reference example.

18. The method of claim 17, wherein the grouping associated with the reference example comprises all reference examples with a same label as the reference example.

19. The method of claim 16, wherein the reference dataset comprises a test dataset used to test a performance of the one or more machine-learned models following a training process.

20. The method of claim 16, wherein the reference dataset comprises a training dataset used to train the one or more machine-learned models, and wherein the weighted performance comprises a weighted loss, and wherein the method further comprises training, by the one or more computing devices, the one or more machine learned models based at least in part on weighted loss.

21. (canceled)

22. The method of claim 17, wherein the parent dataset exhibits a population-level distribution.
